(19) United States
(12) Patent Application Publication
(10) Pub. No.: US 2006/0183812 A1
(43) Pub. Date: Aug. 17, 2006

(54) Adsorptive coating formulation

(76) Inventors: James R. Miller, Roanoke, VA (US); Thomas M. Sisson, Charleston, SC (US); Edward Donald Tolles, Charleston, SC (US); Harry W. Robertson IV, Covington, VA (US)

Correspondence Address: MEADWESTVACO CORPORATION, REGIONAL OFFICE BUILDING, PO BOX 118005, CHARLESTON, SC, 29423-8005, US

(21) Appl. No.: 11/059,223

(22) Filed: Feb. 16, 2005

(51) Int. Cl. A61L 9/04

(52) U.S. Cl. 523/102

(57) ABSTRACT

Novel forms of adsorbent media and, more specifically, novel forms of sub-micron adsorbent media are disclosed that can be coated onto substrates and adsorb vapor-phase contaminants. Such media is disclosed to be activated carbon, which is combined with a dispersant and defoamer, milled to a sub-micron particle size, and mixed with a wax and a binder.

### BACKGROUND OF THE INVENTION

1. Field of the Invention

This invention relates to a material and means for rendering a substrate odor-sorbing. Many odor-sorbing substrates are conveniently converted into packaging for odiferous items, such as some foods (e.g., fish) or chemicals. More particularly, this invention relates to an adsorptive coating formulation for applying on a substrate. Specifically, this invention relates to an improved aqueous-based activated carbon-containing coating formulation comprising sub-micron activated carbon particles and a dispersant which can be applied to a variety of substrates using standard methods and is useful for adsorbing vapor-phase contaminants.

2. Description of Related Art

Activated carbon is one of the most widely accepted materials to adsorb vapor-phase and liquid-phase contaminants. Activated carbon is a microcrystalline, nongraphitic form of carbon that has been processed to increase internal porosity. Activated carbons are characterized by a large specific surface area, typically in the range of 500-2500 m.sup.2/g, which permits its industrial use in the purification of liquids and gases by the adsorption of gases and vapors from gases and of dissolved or dispersed substances from liquids.

Commercial activated carbon has been made from material of plant origin, such as hardwood and softwood, corncobs, kelp, coffee beans, rice hulls, fruit pits, nutshells, and wastes such as bagasse and lignin. Activated carbon also has been made from peat, lignite, soft and hard coals, tars and pitches, asphalt, petroleum residues, and carbon black.

Activation of the organic raw material is accomplished by one of two distinct processes: (1) chemical activation or (2) thermal activation. The effective porosity of activated carbon produced by thermal activation is the result of gasification of the carbon at relatively high temperatures (after an initial carbonization of the raw material), but the porosity of chemically activated products generally is created by chemical dehydration/condensation reactions occurring at significantly lower temperatures. Activated carbons produced by thermal activation are typically more microporous (i.e., pore size no more than 1.8 nanometers); while carbons produced by chemical activation are typically more mesoporous (i.e., pore size in a range of above 1.8 up to 5 nanometers). Pore size distribution is often a controlling factor in adsorption of liquid and gas-phase contaminants.

Carbon black, on the other hand, is relatively non-porous compared with activated carbon. As such, it is not adsorptive and is not used in purification applications. It is typically made by injecting oil into combustion gas flowing through a reactor at about 3000° F. The hydrocarbon is cracked and dehydrogenated to produce agglomerates of nano-scale carbon particles having a quasi-graphitic structure. One of its most common uses is as a black pigment in printing inks.

Most common commercial grades of activated carbon are available in three forms. These are powdered, granular, or shaped (usually, pelletized). Shaped activated carbons are achieved by extrusion of a blend of powdered activated carbon with bentonite clay through a die. The normal choice of die shape produces a cylindrical pellet. Powdered carbons are finely divided particles having a median particle diameter ranging in size from 20 to 50 microns; granular carbons are irregularly shaped particles ranging in size from 0.5 to 4 mm; and pelletized carbons are smooth, hard cylinders typically characterized by diameters ranging from 1 to 4 mm. Powdered carbons are generally used in liquid-phase applications where the carbon is mixed with the liquid being purified and is then separated from the liquid using filtration technology. Granular carbons are used in both vapor and liquid-phase applications where, again, the carbon is held in a canister or large column. Pelletized carbons are generally used in vapor-phase applications where the carbon is held in a canister or large column.

The above forms of activated carbon are good for most applications involving flowing liquid and vapor-phase streams where an activated carbon-filled canister, column, or filtration apparatus can be installed, such as home and municipal water purification, industrial and residential air purification, and purification of in-process streams in food, chemical, and pharmaceutical production processes. For other applications which are not amenable to having equipment for containing the carbon, more convenient forms of activated carbon have been developed. These forms include blends of powdered activated carbon and binder that can be applied directly to a variety of pre-formed substrates, thereby eliminating the need for a canister or column-type device to hold pelletized or granular carbon or a filtration device to capture the powdered carbon. This facilitates the use of the activated carbon in such applications as odor control personal care products, odor control packaging, low-pressure adsorbent monolithic structures used in commercial vapor recovery operations, and adsorbent building materials.

U.S. Pat. Nos. 5,540,916 and 5,693,385 disclose aqueous coating compositions comprising activated carbon particles dispersed in a sodium silicate or polyester binder system. The coating compositions are disclosed to be useful for coating paperboard, resulting in odor-sorbing packaging. Methods of application such as gravure printing, air knife, wire wound rod or blade coating were also disclosed.

Complementing the coating of rigid paperboard substrates, U.S. Pat. No. 6,639,004 B2 discloses a two-step coating process for applying an aqueous activated carbon coating composition onto flexible substrates, such as polyethylene film. Like the coated paperboard, the coated flexible substrates were produced for odor control packaging. The coating comprises activated carbon particles dispersed in a styrene-acrylate binder system. A two-step process is disclosed as a means of increasing the coat weight, and thereby increasing the adsorption capacity of the coated film. A carbon particle size range of 5-40 microns is disclosed.

European Patent Application 0392528A2 describes a porous sheet-type media (such as an open, non-woven fabric) which is coated with odor-adsorbent particles (such as activated carbon) attached via a binder in an aqueous solution. This results in an odor-adsorbing media which has flow-through properties. The particle size of the adsorbent is described to be typically 1-5 microns. The coating is applied through a dip-and-squeeze process.

U.S. Patent Application 20040020359 A2 discloses a vehicular atmosphere cleansing system whereby activated carbon particles are applied to a substrate with a temperature resistant silicone binder for the purpose of adsorbing hydrocarbons. This application teaches that smaller particles have better adsorption capacity. A comparison is made between coatings containing 5 micron particles and 14 micron particles. It is shown that the adsorption capacity for toluene vapor is greater for the coating containing the 5 micron particles.

U.S. Patent Application 20040121681 discloses an alternative method for obtaining a carbon-based coating on a substrate. The carbon-based coating is obtained by coating a porous substrate with a polymer and an activation agent followed by heating to high temperatures (100-300 C.) to carbonize and activate the coating.

The simplified adsorptive coating formulations disclosed in the prior art above have been made with activated carbons having a particle size greater than 1 micron and with little or no dispersant.

Of course, formulations used for high quality printing contain pigments, such as carbon black, which are typically sub-micron and which contain an appreciable amount of dispersant. General formulations are disclosed in U.S. Pat. Nos. 5,630,868, 4,530,961, and 5,281,261. The use of sub-micron particles and a dispersant in printing inks is required to achieve good ink stability and to achieve high quality print appearance throughout long printing runs using standard high-speed printing methods, such as gravure, flexographic, and ink-jet. In general, smaller particles give excellent color strength, saturation, gloss, hiding power, flow, and a stable dispersion, while larger particles result in poor dispersion, plate or cylinder wear, clogged nozzles, poor ink/water balance, printability problems, poor flow, lower hiding power, color fluctuation, and lower gloss.

While carbon black-based formulations embody all these above-mentioned desirable properties, they are not useful for adsorption of contaminants since carbon black itself has relatively little surface area. Activated carbons, on the other hand, have large surface areas for adsorption. As mentioned above, the surface area of activated carbons range from 500 to 2500 m2/gram. However, replacing carbon black with activated carbon in a typical printing ink formulation would not be expected to provide a stable, printer-friendly, adsorptive coating formulation due to the very adsorptive nature of the activated carbon. One skilled in the art would expect high levels of dispersants to cause a substantial challenge since they are relatively low in molecular weight (3,000-20,000 Dalton) and can readily adsorb into activated carbon, thereby plugging pores and minimizing adsorption of target contaminants. Also, the different types of activated carbons have the potential to adsorb different amounts and types of dispersants due to their different pore size distribution. This makes it very difficult to understand what the resultant surface area and pore size distribution of the coating formulation will be based on the surface area and pore size distribution of the activated carbon powder.

Accordingly, it is an object of this invention to provide an improved, high-quality, adsorptive coating formulation for adsorption of vapor-phase contaminants which can utilize a variety of activated carbon types and be applied to a variety of substrates in a cost effective and expeditious manner.

### SUMMARY OF THE INVENTION

The objects of the present invention are met in the novel forms of adsorbent media and, more specifically, novel forms of sub-micron adsorbent media that can be coated onto substrates and adsorb vapor-phase contaminants. For this purpose, the present invention employs activated carbon, which is combined with a dispersant and defoamer, milled to a sub-micron particle size, and mixed with a wax and a binder.

Activated carbon products, such as the Nuchar® products sold by MeadWestvaco Corporation, are milled to a sub-micron particle size, which are dispersable in coatings, inks, or the like and are suitable for application to a variety of substrates such as polyolefin flexible films. The benefit of having sub-micron particles is to improve the kinetics of adsorption, to improve the graphic appearance of the coated product, and improve the runnability of conventional high-speed printing methods such as gravure, flexographic, and ink-jet. It is contemplated that substrates in addition to polyolefin films could also be used such as other types of synthetic films, paperboard, paper, coated paper, laminated paper, cellulosic and synthetic-based non-wovens, metals, ceramics, and rigid plastics. In addition to using conventional high-speed printing methods, such as gravure, flexographic, and ink jet for applying the coating, other methods of application can be used such as air knife, wire round rod, blade coating, spray coating, and dip coating. After application of the adsorptive coating onto the substrate, the coated product can be used “as is” or converted into packages, liner elements, trash bags, pouches, structured media, monolithic structures, building materials or the like suitable for use in many different applications where adsorption of vapor phase contaminants is desired. These applications can include odor adsorption, adsorption of harmful air-borne contaminants which may or may not be odiferous, and recovery of valuable vapor-phase compounds which may or may not be odiferous. Liquid-phase applications can also be contemplated such as the removal of contaminants from aqueous or organic streams, decolorization of colored streams, and recovery of valuable compounds from aqueous or organic streams.

It is therefore an object of this invention to provide an improved, high-quality, adsorptive substrate coating formulation that includes small, sub-micron activated carbon particles and has high surface area for adsorption of vapor-phase contaminants. It is further an object of this invention to provide a an improved, high-quality, adsorptive substrate coating formulation that has a significant amount of adsorptive surface area, relative to loose carbon powder, over a broad range of activated carbon types.

The above objects of the invention are achieved by combining various standard activated carbon powders with a dispersant and defoamer, milling the carbon/dispersant/defoamer solution to achieve a sub-micron particle size, and adding a wax and binder in amounts sufficient to bind the activated carbon particles to a substrate and minimize rub-off. It was surprising to find that even with the elevated level of relatively low molecular weight dispersant, there remained an appreciable activated carbon surface area available for adsorption. Furthermore, over a broad range of activated carbon powder types having widely different pore size distributions, it was surprising that the surface area of the dried coating formulation systematically increased as the surface area of the activated carbon powder used in the coating increased.

### DETAILED DESCRIPTION

The present invention is directed to providing substrates with odor-sorbing properties by the use of activated carbon in an adsorptive coating formulation. In a preferred embodiment, the adsorptive coating formulation is prepared using various activated carbons to provide a coating with an activated carbon surface area of at least 100 m2/g and a median particle size no greater than 1 micron.

The types of activated carbons used included thermally activated wood, coal, and coconut-based carbons and chemically-activated wood-based carbons. Thermal activation agents may include steam, oxygen, and carbon dioxide. Most preferred is steam. Chemical activation agents may include: alkali metal hydroxides, carbonates, sulfides, and sulfates; alkaline earth carbonates, chlorides, sulfates, and phosphates; phosphoric acid; polyphosphoric acid; pyrophosphoric acid; zinc chloride; sulfuric acid; and oleum. Preferred among these are phosphoric acid and zinc chloride. Most preferred is phosphoric acid.

Although granular or pelletized activated carbons could have been used, the powdered form was utilized since it is readily available and requires less milling time to achieve sub-micron particle size.

The thermally activated carbons included MeadWestvaco TAC-600 wood-based carbon (available in powdered form), Pica PW-2 coconut-based carbon (available in powdered form) and Calgon CPG coal-based carbon (available in granular form, but ground to a powder for the present invention). The chemically-activated carbons were all produced by MeadWestvaco in powder form. These included Nuchar® SA-20, SA-400, TC-400, SA-1500, and RGC.

In addition, a powdered carbon black commonly used in printing applications was used for comparison. It was Black Pearls 410 made by Cabot.

In addition to the activated carbon and carbon black, other raw materials may include a binder, defoamer, wax, dispersant, ammonium hydroxide, solvent, water and various combinations thereof. The binder chosen was an emulsion styrene-acrylate coplymer, Jonrez I-988 produced by MeadWestvaco (38% solids). The wax was a polyethylene emulsion, Jonrez W-2320 produced by MeadWestvaco (25% solids). The defoamer was an organic petroleum derivative, FoamBlast 370 produced by Lubrizol (20% solids). The dispersant was a styrene acrylic acid copolymer, Jonrez H-2702 produced by MeadWestvaco (100% solids). Binders for water-based pigmented coatings are typically emulsion or water soluble polymers. The compositions are varied by the selection of monomers and varied to optimize adhesion, water resistance, barrier, appearance, and other performance properties. Binder properties often include the ability to disperse insoluble materials. Binders most often contain surfactants or polymeric resins that surround insoluble particulates in an aqueous media increasing steric hinderance or creating electrostatic repulsions between the associated particles. Generally waxes are natural or synthetic and available as emulsions, dispersions, or powders. Waxes impart rub, mar, and water resistance. Natural waxes are either paraffin or Carnuba types and synthetic waxes are polyethylene or polytetrafluoroethylene (PTFE). Dispersants for pigments such as carbon black are either polymeric or surfactants. The mechanism for dispersing pigments is via electrostatic and/or steric repulsions. Polymeric dispersants are either low molecular weight (3000-20,000 Daltons) styrene-acrylic acid copolymers or colloidal dispersions. The chemistry of defoamers is based on aromatic or aliphatic petroleum derivatives, aliphatic oils, mineral oils, or silicone. Defoamers work through two mechanisms depending on the chemistry of the overall system. Some solubilize the surface active surfactants in the system, rapidly destroying the monolayer or lamella at the air-liquid interface. A second mechanism is by dramatically lowering the surface tension of the liquid destabilizing bubbles formed at the surface. In addition to defoamers, solvents are also used to lower the surface tension of aqueous-based coatings. Typical solvents include alcohols and glycols with one or more hydroxyl groups, ethers, esters, hydrocarbons, aromatics, and mineral spirits.

Table I shows the percentages of raw materials found in the adsorptive coating formulation and a typical carbon black printing ink.

TABLE I
Raw Materials

| | | | Amount in Typical Carbon | |
|---|---|---|---|---|
| | Amount in Adsorptive | | Black Printing Ink | |
| Raw | Coating Formulation | | Formulation | |
| Material | (% wet basis) | (% dry basis) | (% wet basis) | (% dry basis) |
| Carbon | 23.2 | 62.8 | 15.0 | 48.9 |
| (activated | | | | |
| carbon or | | | | |
| carbon | | | | |
| black) | | | | |
| Water | 43 | 0 | 28.0 | 0 |
| Binder | 7.2 | 7.3 | 18.0 | 23.4 |
| Dispersant | 7.3 | 19.8 | 29.0 | 22.8 |
| Defoamer | 0.3 | 0.2 | 0.3 | 0.06 |
| Ammo- | 4.2 | 0 | 3.7 | 0 |
| nium | | | | |
| Hydroxide | | | | |
| Wax | 14.8 | 10.0 | 6.0 | 4.9 |

To produce a coating using each carbon type on the laboratory scale (Process A), the raw materials were combined in a blender available from Waring and blended for 20 minutes. In this step, very little particle size reduction occurred, only physical blending. The blended materials were transferred to a Szegvari Attritor System ball mill, using 1.0-1.6 mm zirconium beads, where the carbon was milled for 12 to 30 hours to obtain particle sizes less than 1 micron. An alternative method for producing a coating (Process B) involved first blending water, dispersant, ammonium hydroxide, and defoamer in the Waring blender for 20 minutes to solubilize the dispersant. This mixture was then transferred to a shot mill (Eiger) where the carbon was added, along with additional defoamer and ammonium hydroxide. Shot milling proceeded for 30 minutes. Binder, wax and additional dispersant was then blended into the shot mill product. The only deviation from the formulation given in Table I was that the total level of dispersant was 14.6%, the total level of binder was 7.3%, and the total level of wax was 7.4% (all on a wet basis).

Following milling, the particle size distribution of the coating was measured (Beckman Coulter N4 Plus Submicron Particle Size Analyzer) to ensure that its median size was less than 1 micron. Measurements were also made of the viscosity (using #2 Zahn Cup). Viscosities of all coating formulations ranged from 18-28 seconds. Depending on the end-use, drawdowns were made with a an RK Print-Coat Instruments automated coater using a #1 bar (6 micron thick wet coating) on either commercial polyethylene film or glass plates and dried with heated air. Drawdowns were made on glass so that the dried coating could be removed and tested for adsorption capacity and surface area. The targeted coat weight of the dried draw down was 4-10 grams/m2. This is a reasonable coat weight for most applications.

Surface area of the dried coating removed from the glass plates was measured with was measured with a Micromeritics ASAP 2010 Surface Area and Porosimetry System.

Adsorption capacity of the dried coating removed from the glass plates was measured using a common odorant, Dimethyldisulfide (DMDS). DMDS is an odor component of garlic, human waste, and some industrial process such as the Kraft pulping process. DMDS is extremely odorous, having an odor threshold of 0.001 ppm. This is much lower than other common odorants, such as ammonia which has an odor threshold of 10 ppm. Adsorption capacities of the various coating formulations were measured by headspace analysis using a Hewlett Packard 5890 gas chromatograph with a Perkin Elmer HS40 headspace sampler. Quantities of the dried coating film ranging from 10 to 160 mg were introduced into a series of headspace vials. Sufficient DMDS liquid was then injected into the vials to produce a vapor phase concentration of 2.5% by volume in the absence of any adsorbent. GC analysis was conducted to determine the concentration of DMDS in the vial after equilibration with the adsorbent coating. The amount adsorbed was determined by difference, and the amount adsorbed per gram of coating was calculated.

Examples are provided which illustrate the benefit of milling the coating so that the carbon is sub-micron, and the beneficial impact of carbon type on surface area of the coating.

### EXAMPLE 1

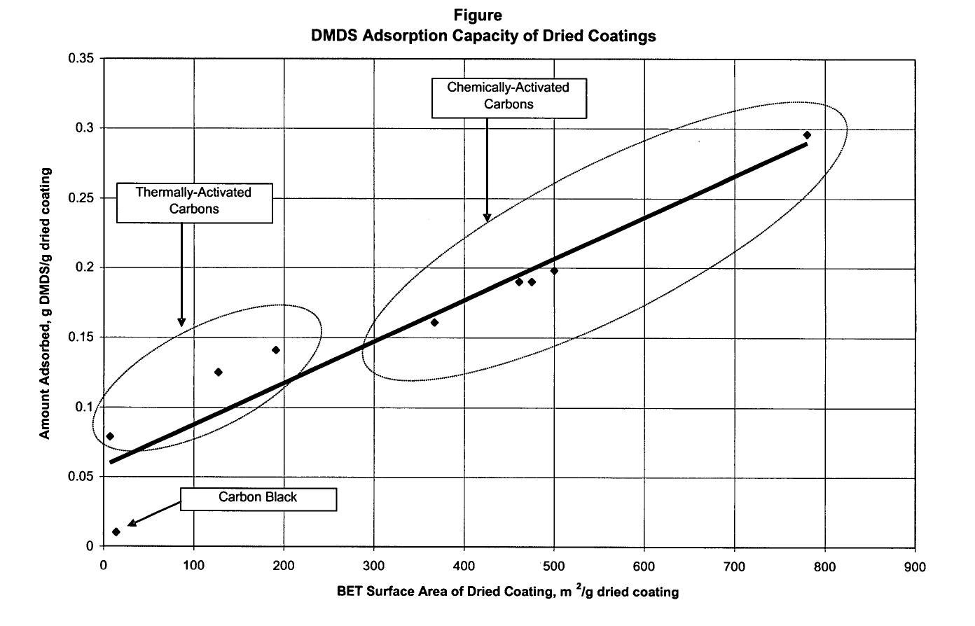

A sample of the coating made by Process B with Nuchar TC-400 was drawn down onto polyethylene film using a #1 bar. This sample had a median particle size of 0.6 microns. Additionally, a sample of coating made with Nuchar TC-400 produced by just blending the components without milling was also drawn down onto polyethylene film using a #4 bar (36 micron thick wet coating) in a similar manner. This sample was much more coarse, having a median particle size of 15 microns. A #1 bar could not be used to draw down the unmilled coating because the coarse particles would not allow the coating to pass underneath the bar. In addition to visual appearance, the so-called Scotch Tape test was used to compare the adhesion of the coatings. Digital photographs were taken of the draw downs and are shown in FIG. 1. It is clear that the coating made by Process B had much better coverage, appearance, and adhesion properties than the unmilled coating. Although there was slight removal of the coating made by Process B with the Scotch Tape, there is much more remaining relative to the unmilled coating.

### EXAMPLE 2

Samples of the coating were made using Process A using each activated carbon types and carbon black previously mentioned. The median particle size of the coatings was measured and the coatings were drawn down onto glass plates, dried, and removed from the plates for analysis of BET surface area and DMDS adsorption capacity. The BET surface area of the loose carbon powder was also measured and recorded. By knowing the surface area of the dried coating and the loose carbon powder, and estimating a carbon content in the dried coating, the fraction of surface area remaining in the carbon (F) was calculated by the following equation:

$$Fraction of Surface Area Remaining in Carbon ( F ) = \frac{Surface Area of Dried Coating}{0.628 * Surface Area Of Loose Powder}$$

The “0.628” factor is based on the estimate that the dried coating contains 62.8% carbon. The results are shown in Table II and FIG. 2.

TABLE II
Properties of Carbon Powder and Coatings for Example 2

| | BET Surface Area | | | Median Particle | |
|---|---|---|---|---|---|
| | Loose Carbon | | | Diameter of | Adsorption Capacity of DMDS |
| | Powder | Dried Coating | Fraction of Surface | Coating | at 1000 ppm DMDS |
| Carbon Type | (m²/g) | (m²/g) | Area Remaining = F | (microns) | (g DMDS/g Dried Coating) |
| MWV Nuchar | 2219 | 780 | 0.56 | 0.405 | 0.296 |
| SA-1500 | | | | | |
| MWV Nuchar | 1463 | 500 | 0.54 | 0.825 | 0.198 |
| RGC | | | | | |
| MWV Nuchar | 1659 | 475 | 0.45 | 0.475 | 0.190 |
| TC-400 | | | | | |
| MWV Nuchar | 1633 | 461 | 0.45 | 0.320 | 0.190 |
| SA-20 | | | | | |
| MWV Nuchar | 1604 | 367 | 0.37 | 0.140 | 0.161 |
| SA-400 | | | | | |
| Pica | 1140 | 191 | 0.27 | 0.750 | 0.141 |
| PW-2 | | | | | |
| Calgon | 891 | 127 | 0.23 | 0.850 | 0.125 |
| CPG | | | | | |
| MWV | 586 | 7 | 0.02 | 0.450 | 0.079 |
| TAC-600 | | | | | |
| Cabot | | 14 | | 0.190 | 0.010 |
| Black Pearls | | | | | |
| Carbon Black | | | | | |

The results clearly show that the activated carbon—based formulations are significantly more adsorptive than the formulation made with carbon black. They data also show that the adsorption capacity of the present invention correlates strongly with the surface area of the loose carbon powder and the surface area of the dried carbon coating over a wide range of activated carbon types. This is surprising given the great difference in pore size distributions of the different carbon types. Furthermore, the data also show that as surface area of the loose carbon powder or dried carbon coating increases, the fraction of surface area remaining in the carbon increases. Based on the present invention, a reasonable lower limit for F is 0.20, equivalent to a lower limit of BET surface area in the dried coating of approximately 100 m2/g. This is further equivalent to a DMDS adsorption capacity of 0.1 g/g dried coating, which is reasonable for vapor-phase adsorption.

What is claimed is:

1. An adsorptive coating formulation useful for rendering a substrate odor-sorbing comprising a mixture of activated carbon characterized by a median particle size of less than 1 micron and an aqueous binder system characterized by an amount of a dispersant sufficient to suspend the activated carbon particles, said coating formulation, upon drying, further characterized by a dry basis BET Surface Area of greater than 100 m2/g.

2. The adsorptive coating formulation of claim 1 further comprising a solids content in the range from about 25 to about 45%, said solids comprising from about 20 to about 95% activated carbon and from about 5 to about 80% binder, and about 5 to about 80% dispersant.

3. The adsorptive coating formulation of claim 2 wherein the solids content further comprises about 4-12% wax and about 0.05-1% defoamer.

4. The adsorptive coating formulation of claim 2 further comprising about 0.5 to about 10% solvent.

5. The adsorptive coating formulation of claim 2 wherein the binder is a member selected from the group consisting of vinylic emulsion and colloidal copolymers with monomer compositions selected from a group consisting of acrylic acid, methacrylic acid, methyl methacrylate, ethyl methacrylate, styrene, n-propyl methacrylate, n-butyl methacrylate, isopropyl methacrylate, isobutyl methacrylate, n-amyl methacrylate, n-hexyl methacrylate, isoamyl methacrylate, 2-hydroxylethyl methacrylate, 2-hydroxypropyl methacrylate, N,N-dimethylaminoethyl methacrylate, N,N-diethylaminoethyl methacrylate, t-butylaminoethyl methacrylate, 2-sulfoethyl methacrylate, trifluoroethyl methacrylate, glycidyl methacrylate, benzyl methacrylate, allyl methacrylate, 2-n-butoxyethyl methacrylate, 2-chloroethyl methacrylate, sec-butyl-methacrylate, tert-butyl methacrylate, 2-ethybutyl methacrylate, cinnamyl methacrylate, crotyl methacrylate, cyclohexyl methacrylate, cyclopentyl methacrylate, 2-ethoxyethyl methacrylate, furfuryl methacrylate, hexafluoroisopropyl methacrylate, methallyl methacrylate, 3-methoxybutyl methacrylate, 2-methoxybutyl methacrylate, 2-nitro-2 methylpropyl methacrylate, n-octylmethacrylate, 2-ethylhexyl methacrylate, 2-phenoxyethyl methacrylate, 2-phenylethyl methacrylate, phenyl methacrylate, propargyl methacrylate, tetrahydrofurfuryl methacrylate, tetrahydropyranyl methacrylate, methyl acrylate, ethyl acrylate, n-propyl acrylate, isopropyl acrylate, n-butyl acrylate, n-decyl acrylate, 2-ethylhexyl acrylate, salts of methacrylic acid, methacrylonitrile, methacrylamide, N-methylmethacrylamide, N-ethylmethacrylamide, N,N-diethymethacrylamide, N,N-dimethylmethacrylamide, N-phenyl-methacrylamide, methacrolein, salts of acrylic acid, acrylonitrile, acrylamide, methyl alpha-chloroacrylate, methyl 2-cyanoacrylate, N-ethylacrylamide, N,N-diethylacrylamide acrolein, vinyl acetate, vinyl chloride, vinyl pyridine, vinyl pyrollidone, sodium crotonate, methyl crotonate, crotonic acid, maleic anhydride, and combinations thereof.

6. The adsorptive coating formulation of claim 2 wherein the glass transition temperature for the binder is in the range of about −40 deg C. to about 100 deg C.

7. The adsorptive coating formulation of claim 2 wherein the binder also functions as a dispersant for the activated carbon when the binder contains surfactants or polymeric resins.

8. The adsorptive coating formulation of claim 2 wherein the molecular weight for the dispersant is in the range of 3000-20,000 Daltons.

9. The adsorptive coating formulation of claim 2 wherein the binder comprises a member selected from the group consisting of vinylic emulsion and colloidal copolymers.

10. The adsorptive coating formulation of claim 2 wherein the dispersant of claim 2 is a member selected from the group consisting of surfactant and styrene-acrylic acid copolymer.

11. The adsorptive coating formulation of claim 3 wherein the wax is a member of the group consisting of natural and synthetic waxes.

12. The adsorptive coating formulation of claim 3 wherein the defoamer is a member selected from the group consisting of aromatic or aliphatic petroleum derivatives, aliphatic oils, mineral oils, or silicone.

13. The adsorptive coating formulation of claim 4 wherein the solvent is a member selected from the group consisting of alcohols and glycols with one or more hydroxyl groups, ethers, esters, hydrocarbons, aromatics, and mineral spirits.

14. The adsorptive coating formulation of claim 1 wherein the substrate is selected from the group of substrates consisting of synthetic films, paperboard, paper, coated paper, laminated paper, cellulosic and synthetic-based non-wovens, metals, ceramics, and rigid plastics.

15. The adsorptive coating formulation of claim 14 wherein the synthetic film is a member of the group consisting of polyester films and polyolefin films.
